(19) United States
(12) Patent Application Publication
(10) Pub. No.: US 2023/0133201 A1
(43) Pub. Date: May 4, 2023

(54) METHODS AND MATERIALS FOR DISSEMINATING A PROTEIN THROUGHOUT THE CENTRAL NERVOUS SYSTEM

(71) Applicant: REMOTOR THERAPEUTICS, INC., San Diego, CA (US)

(72) Inventor: Karl K. JOHE, Miami, FL (US)

(21) Appl. No.: 17/910,292

(22) PCT Filed: Mar. 10, 2021
(86) PCT No.: PCT/US2021/021757

§ 371 Date: Sep. 8, 2022

Related U.S. Application Data
(60) Provisional application No. 62/987,937, filed on Mar. 11, 2020.

(51) Int. Cl. A61K 35/30; A61P 25/28; C12N 15/86; C12N 9/14

(52) U.S. Cl. CPC A61K 35/30; A61P 25/28; C12N 15/86; C12N 9/14; C12Y 310/01001

(57) ABSTRACT

The present disclosure provides methods for disseminating a protein such as an enzyme in a central nervous system tissue by depositing neural stem cells or neural precursor cells that comprise a nucleotide that encodes the enzyme in a white matter tract, wherein the neural stem cells or neural precursor cells secrete the enzyme. Also provided are methods of treating a disease or disorder that is due to a lack or deficiency of a lysosomal enzyme in a brain in a subject in need thereof by administering neural stem cells or neural precursor cells that express an enzyme that is lacking or deficient in the brain of the subject to one or more white matter tracts in the brain.

### CROSS REFERENCE TO RELATED APPLICATIONS

This application is a national stage application of International Patent Application No. PCT/US2021/021757, filed on Mar. 10, 2021, which claims the benefit of and priority to U.S. patent application Ser. No. 62/987,937, filed on Mar. 11, 2020, each of which is hereby incorporated by reference in its entirety.

### FIELD

The present disclosure relates to methods for disseminating a protein such as an enzyme in a central nervous system tissue by depositing neural stem cells or neural precursor cells that comprise a nucleotide that encodes the protein in a white matter tract, wherein the neural stem cells or neural precursor cells secrete the enzyme.

### BACKGROUND

Lysosomes are organelles found in most eukaryotic cells and are commonly referred to as a cell's recycling center because they process and digest unwanted material into substances that cells can use. Each lysosome is surrounded by a membrane that maintains an acidic environment within the interior via a proton pump. Lysosomes contain a wide variety of hydrolytic enzymes (acid hydrolases) that break down macromolecules such as nucleic acids, proteins, and polysaccharides.

Lysosomal disorders (LSDs) occur when a particular lysosomal enzyme is deficient or missing resulting in the accumulation of macromolecules in the cell. LSDs include inborn monogenic diseases such as mucopolysaccharidosis (MPS) which is caused by deficient activity of a single lysosomal enzyme. However, treatment options for LSDs remain limited. For example, enzyme replacement therapies have been effective for correcting enzyme deficiencies in peripheral organs but not for central nervous system. Further, infusion of an enzyme by systemic routes cannot cross the blood brain barrier sufficiently to be clinically effective in the brain. Consequently, there is a need for therapeutic approaches to treat diseases and disorders in the central nervous system (CNS) caused by a missing protein such as a missing enzyme implicated in a LSD.

### SUMMARY

The present disclosure provides methods for disseminating a protein such as an enzyme in a central nervous system tissue (e.g., the brain or spinal cord) by depositing neural stem cells or neural precursor cells that comprise a nucleotide that encodes the protein in a white matter tract, wherein the neural stem cells or neural precursor cells secrete the protein. Advantageously, the neural stem cells or neural precursor cells are migratory and move along the white matter tracts while secreting the missing protein that is then taken up by cells of the central nervous system.

In some embodiments of each or any of the above- or below-mentioned embodiments, the enzyme is a lysosomal enzyme.

In some embodiments of each or any of the above- or below-mentioned embodiments, the lysosomal enzyme is N-sulfoglucosamine sulfohydrolase (SGSH).

In some embodiments of each or any of the above- or below-mentioned embodiments, the neural stem cells are migratory neural stem cells.

In some embodiments of each or any of the above- or below-mentioned embodiments, the neural stem cells are capable of at least 60 cell doublings.

In some embodiments of each or any of the above- or below-mentioned embodiments, the neural stem cells are human neural stem cells.

In some embodiments of each or any of the above- or below-mentioned embodiments, the neural stem cells are neural progenitors derived from induced pluripotent stem cells.

In some embodiments of each or any of the above- or below-mentioned embodiments, the neural stem cells are adherent.

In some embodiments of each or any of the above- or below-mentioned embodiments, the central nervous system tissue is a brain.

In some embodiments of each or any of the above- or below-mentioned embodiments, the central nervous system tissue is a spinal cord.

In some embodiments of each or any of the above- or below-mentioned embodiments, the neural stem cells are derived from a fetal cortical tissue.

In some embodiments of each or any of the above- or below-mentioned embodiments, the neural stem cells are conditionally immortalized with cMyc-ER

In some embodiments of each or any of the above- or below-mentioned embodiments, the neural stem cells are programmed to differentiate into neurons, oligodendrocytes, and astrocytes.

The present disclosure also provides methods of treating a neurodegenerative disease that is due to a lack of a protein such as a lysosomal enzyme in a central nervous system tissue (e.g., the brain) in a subject in need thereof by administering neural stem cells that express a protein that is deficient in the central nervous system of the subject to one or more white matter tracts in the central nervous system.

In some embodiments of each or any of the above- or below-mentioned embodiments, the neurodegenerative disease is MPS IIIA (MPS3a).

In some embodiments of each or any of the above- or below-mentioned embodiments, the enzyme is a lysosomal enzyme.

In some embodiments of each or any of the above- or below-mentioned embodiments, the lysosomal enzyme is N-sulfoglucosamine sulfohydrolase (SGSH).

In some embodiments of each or any of the above- or below-mentioned embodiments, the neural stem cells are deposited by intracerebral transplantation.

In some embodiments of each or any of the above- or below-mentioned embodiments, the central nervous system tissue is a brain.

In some embodiments of each or any of the above- or below-mentioned embodiments, the step of depositing the neural stem cells into the central nervous system tissue comprises injections of the cells into white matter tracts of the corona radiata, internal capsule, corpus callosum, and cerebellum bilaterally.

In some embodiments of each or any of the above- or below-mentioned embodiments, eight injection tracks are used to deposit the neural stem cells into white matter tracts of corona radiata, internal capsule, corpus callosum, and cerebellum bilaterally.

In some embodiments of each or any of the above- or below-mentioned embodiments, the injections are made in two stages.

In some embodiments of each or any of the above- or below-mentioned embodiments, the first stage comprises injections of the neural stem cells at six tracks into the cerebrum in supine position followed by injections of the neural stem cells at two cerebellar tracks about 2-8 weeks after the injections of the first stage.

In some embodiments of each or any of the above- or below-mentioned embodiments, the neural stem cells comprise a vector comprising: (a) a human N-sulfoglucosamine sulfohydrolase (SGSH) coding sequence; and (b) an EF1A promoter, wherein the vector is a lentiviral vector, and wherein neural stem cells comprising the vector are characterized by activity of expressed SGSH protein that is between about 20- to about 300-fold higher than physiological activity of SGSH protein.

In some embodiments of each or any of the above- or below-mentioned embodiments, the human SGSH coding sequence comprises an artificial secretory signal sequence, wherein the artificial secretory signal sequence increases SGSH secretion by between about 20% to about 200% as compared to the native secretory signal sequence of SGSH.

In some embodiments of each or any of the above- or below-mentioned embodiments, the human SGSH coding sequence is a recombinant human SGSH coding sequence.

### BRIEF DESCRIPTION OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIG. 1: Hypothesized cell injection routes to maximize the cell spreading with fewest penetrations bilaterally. The blue dots represent the most distal cell deposition in each injection track.

FIG. 2: SGSH activity in HK532.SGSH. HK532 cells were transduced with a lentiviral vector expressing human SGSH coding sequence under CAG promoter and expanded. Conditioned media and cell lysates from the virally transduced and no virus control cultures were measured for SGSH activity.

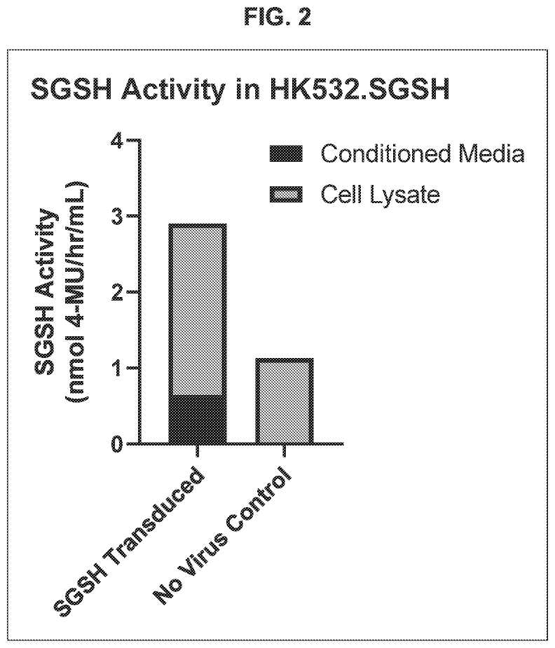

FIG. 3: SGSH activity in MPS3a.SGSH. hNSC culture was derived from iPSC of a MPS IIIA patient. The cells were transduced with a lentiviral (LV) vector expressing a recombinant form of human SGSH optimized for secretion and endocytosis. Using EF1A promoter in a third generation lentiviral vector and optimized transduction conditions resulted in significantly higher LV titer and SGSH expression.

FIG. 4: Infusion in dogs. Three healthy adult dogs were infused with 10, 15, and 20 mL of cell suspension buffer using the clinically intended stereotaxic platform, Z-drive, cannula assembly, and infusion pump. The dog with 10 mL infusion volume fully recovered, thus setting the maximum tolerated infusion volume to 10 mL in dog or 10% of the brain volume.

### DETAILED DESCRIPTION

Lysosomal disorders (LSDs) such as mucopolysaccharidosis (MPS) are inherited, monogenic, multisystem, progressive metabolic diseases caused by a missing or deficient lysosomal enzyme. At least 75% of LSDs present with the most devastating effect on a person's central nervous system (CNS), cognitive and motor functions. There are no curative therapies to treat the underlying disease. Indeed, enzyme therapies on the market or in development fail to cross the blood brain barrier and only treat systemic symptomatology. As such, the prognosis for patients with many types of LSD remains poor and the life expectancy of LSD patients is severely limited.

The inventor has found that human neural stem cells (hNSC) or neural precursor cells including neural progenitor cells that express a recombinant protein can be transplanted directly into a white matter tract where they migrate along the tract and express the protein such as an enzyme in a continuous and sustained manner. Advantageously, the neural stem cells or neural precursor cells may be used in the brain or spinal cord for chronic delivery of a missing or deficient protein. The missing or deficient protein may be specifically taken up and accumulated within cells of the central nervous system including neurons and glia. The neural stem cells or neural precursor cells disclosed herein may also be used to treat and/or prevent many monogenic diseases that affect the CNS such as MPS IIIA.

The neural stem cells for use in the methods of the present disclosure have a migratory property, especially when placed in a white matter tract. In an embodiment, the cells are deposited in white matter tracts bilaterally across the corona radiata, internal capsule, corpus callosum, and/or cerebellar tracts to achieve maximum cell spreading with a minimum set of cannula penetrations.

For the purposes of this disclosure, the terms “neural progenitor cell” and “neural precursor cell” mean a cell that can generate progeny that are either neuronal cells (such as neuronal precursors or mature neurons) or glial cells (such as glial precursors, mature astrocytes, or mature oligodendrocytes). Typically, the cells express some of the phenotypic markers that are characteristic of the neural lineage.

### Neural Stem Cells and Precursor Cells

Neural stem cells and precursor cells are provided that comprise an exogenous polynucleotide sequence that codes for a missing or deficient protein such as an enzyme in a disease or disorder that effects the central nervous system. The neural stem cells and precursor cells are preferably stable and do not differentiate in a culture even after more than 60 cell doublings. The neural stem cells may be human neural stem cells including, for example, fetal human neural stem cells.

In an embodiment, the neural stem cell or precursor cell comprises a SGSH coding sequence, the enzyme missing in MPS IIIA. The coding sequence is preferably stably incorporated into the chromosome of a conditionally immortalized human neural stem cell line (hkNSC) via a lentiviral vector.

The present disclosure provides a vector comprising: (a) a human SGSH coding sequence; and (b) an EF1A promoter, wherein the vector is a lentiviral vector (e.g., a third generation lentiviral vector), and wherein cells comprising the vector are characterized by activity of expressed SGSH protein that is between about 20- to about 300-fold higher than physiological activity of SGSH protein. In an embodiment, the cells comprising the vector are characterized by an activity of expressed SGSH protein that is between about 20- to about 30-fold higher than the physiological activity of SGSH protein. In an embodiment, the cells comprising the vector are characterized by an activity of expressed SGSH protein that is between about 30- to about 40-fold higher than the physiological activity of SGSH protein. In an embodiment, the cells comprising the vector are characterized by an activity of expressed SGSH protein that is between about 40- to about 50-fold higher than the physiological activity of SGSH protein. In an embodiment, the cells comprising the vector are characterized by an activity of expressed SGSH protein that is between about 50- to about 60-fold higher than the physiological activity of SGSH protein. In an embodiment, the cells comprising the vector are characterized by an activity of expressed SGSH protein that is between about 60- to about 70-fold higher than the physiological activity of SGSH protein. In an embodiment, the cells comprising the vector are characterized by an activity of expressed SGSH protein that is between about 70- to about 80-fold higher than the physiological activity of SGSH protein. In an embodiment, the cells comprising the vector are characterized by an activity of expressed SGSH protein that is between about 80- to about 90-fold higher than the physiological activity of SGSH protein. In an embodiment, the cells comprising the vector are characterized by an activity of expressed SGSH protein that is between about 90- to about 100-fold higher than the physiological activity of SGSH protein. In an embodiment, the cells comprising the vector are characterized by an activity of expressed SGSH protein that is between about 100- to about 150-fold higher than the physiological activity of SGSH protein. In an embodiment, the cells comprising the vector are characterized by an activity of expressed SGSH protein that is between about 150- to about 200-fold higher than the physiological activity of SGSH protein. In an embodiment, the cells comprising the vector are characterized by an activity of expressed SGSH protein that is between about 200- to about 250-fold higher than the physiological activity of SGSH protein. In an embodiment, the cells comprising the vector are characterized by an activity of expressed SGSH protein that is between about 250- to about 300-fold higher than the physiological activity of SGSH protein.

In some embodiments, the human SGSH coding sequence comprises an artificial secretory signal sequence, wherein the artificial secretory signal sequence increases SGSH secretion by between about 20% to about 200% as compared to the native secretory signal sequence of SGSH. In an embodiment, the artificial secretory signal sequence increases SGSH secretion by between about 20% to about 30% as compared to the native secretory signal sequence of SGSH. In an embodiment, the artificial secretory signal sequence increases SGSH secretion by between about 30% to about 40% as compared to the native secretory signal sequence of SGSH. In an embodiment, the artificial secretory signal sequence increases SGSH secretion by between about 40% to about 50% as compared to the native secretory signal sequence of SGSH. In an embodiment, the artificial secretory signal sequence increases SGSH secretion by between about 50% to about 60% as compared to the native secretory signal sequence of SGSH. In an embodiment, the artificial secretory signal sequence increases SGSH secretion by between about 60% to about 70% as compared to the native secretory signal sequence of SGSH. In an embodiment, the artificial secretory signal sequence increases SGSH secretion by between about 70% to about 80% as compared to the native secretory signal sequence of SGSH. In an embodiment, the artificial secretory signal sequence increases SGSH secretion by between about 80% to about 90% as compared to the native secretory signal sequence of SGSH. In an embodiment, the artificial secretory signal sequence increases SGSH secretion by between about 90% to about 100% as compared to the native secretory signal sequence of SGSH. In an embodiment, the artificial secretory signal sequence increases SGSH secretion by between about 100% to about 150% as compared to the native secretory signal sequence of SGSH. In an embodiment, the artificial secretory signal sequence increases SGSH secretion by between about 150% to about 200% as compared to the native secretory signal sequence of SGSH.

In some embodiments, the human SGSH coding sequence is a recombinant human SGSH coding sequence. In some embodiments, the recombinant human coding SGSH coding sequence comprises a reduced number of glycosylation sites by introduction of one or more N264Q substitutions, wherein the introduction of one or more N264Q substitutions enhances endocytosis by target cells of the vector as compared to a vector comprising a recombinant human coding SGSH coding sequence comprising no N264Q substitutions.

In some embodiments, between about 10% to about 90% of the expressed SGSH protein is secreted over 24 hours. In an embodiment, between about 10% to about 20% of the expressed SGSH protein is secreted over 24 hours. In an embodiment, between about 20% to about 30% of the expressed SGSH protein is secreted over 24 hours. In an embodiment, between about 30% to about 40% of the expressed SGSH protein is secreted over 24 hours. In an embodiment, between about 40% to about 50% of the expressed SGSH protein is secreted over 24 hours. In an embodiment, between about 50% to about 60% of the expressed SGSH protein is secreted over 24 hours. In an embodiment, between about 60% to about 70% of the expressed SGSH protein is secreted over 24 hours. In an embodiment, between about 70% to about 80% of the expressed SGSH protein is secreted over 24 hours. In an embodiment, between about 80% to about 90% of the expressed SGSH protein is secreted over 24 hours.

The present disclosure provides a vector optimized by (i) substituting the native secretory signal sequence of SGSH with an artificial, more efficient signal sequence to increase SGSH secretion, and (ii) reducing glycosylation sites in the SGSH coding sequence by N264Q substitution to enhance endocytosis by target cells. In an embodiment, the vector utilizes a third generation LV to increase its titer (VectorBuilder, Chicago).

In an embodiment, cells transfected with the vector show an SGSH activity that is about 10-fold, 20-fold, 30-fold, 40-fold, 50-fold, 60-fold, 70-fold, 80-fold, 90-fold, or 100-fold higher than the physiological activity of SGSH protein. In a further embodiment, between about 20% to about 50% of expressed SGSH protein is secreted (e.g., into conditioned media) over twenty-four hours. In another embodiment, about 10%, 20%, 30%, 40%, 50%, 60%, 70%, 80%, 90% or more of expressed SGSH protein is secreted over twenty-four hours.

The present disclosure also provides methods of making a neural stem cell or neural precursor cell comprising an exogenous polynucleotide sequence that codes for a missing or deficient protein in the central nervous system by obtaining one or more human neural stem cells or precursor cells; plating the one or more neural stem cells or precursor cells on a tissue culture treated dish precoated with poly D-lysine and fibronectin; culturing the one or more neural stem cells or precursor cells in a growth medium; expanding the one or more neural stem cells to produce a population of expanded neural stem cells or precursor cells; infecting a neural stem cell or precursor cell with a viral vector that encodes an immortalizing gene; and infecting the neural stem cells or precursor cells previously exposed to immortalizing construct with another vector that encodes the missing or deficient protein. Such immortalized neural stem cells or precursor cells and methods of making the neural cells are disclosed in U.S. Pat. No. 7,544,511.

In one embodiment, the neural stem cells or neural precursor cells are multipotent such that each cell has the capacity to differentiate into a neuron, astrocyte or oligodendrocyte. In another embodiment, the neural stem cells or neural precursor cells are bipotent such that each cell has the capacity to differentiate into two of the three cell types of the CNS. In another embodiment, the neural stem cells or neural precursor cells include at least bipotent cells generating both neurons and astrocytes or oligodendrocytes and astrocytes in vitro and include at least unipotent cells generating neurons, oligodendrocytes, or astrocytes in vivo.

Growth conditions can influence the differentiation direction of the cells toward one cell type or another, indicating that the cells are not committed toward a single lineage. In culture conditions that favor neuronal differentiation, cells, particularly from a human CNS, are largely bipotent for neurons and astrocytes and differentiation into oligodendrocytes is minimal. Thus, the differentiated cell cultures of the disclosed methods may give rise to neurons and astrocytes.

In an embodiment, the neural stem cells or neural precursor cells are isolated from the CNS. As used herein, the term “isolated” with reference to a cell refers to a cell that is in an environment different from that which the cell naturally occurs (e.g., where the cell naturally occurs in an organism) and the cell is removed from its natural environment.

Neural stem cells or neural precursor cells may be isolated from an area that is naturally neurogenic for a desired population of neurons and from embryonic, fetal, post-natal, juvenile or adult tissue. The desired population of cells may include the cells of a specific neuronal phenotype that can replace or supplement such phenotype lost or inactive in the course of disease progression. In an embodiment, the neural progenitor cells are isolated from the subventricular zone (SVZ) or the subgranular zone of the dentate gyrus (DG). In preferred embodiments, the neural progenitor cells are isolated from the spinal cord in which neurogenesis of ventral motor neurons is substantial and obtained at a gestational age of human fetal development during which neurogenesis of ventral motor neurons is substantial.

Accordingly, in an embodiment, neural stem cells or neural precursor cells are isolated from the spinal cord at a gestational age of about 6.5 to about 20 weeks. Preferably, neural stem cells are isolated from the spinal cord at a gestational age of about seven to about nine weeks. In another embodiment, the neural stem cells are isolated from embryonic spinal cord tissue. In yet another embodiment, neural stem cells are isolated from a human. It should be appreciated that the proportion of the isolatable neural stem cell population can vary with the age of the donor. Expansion capacity of the cell populations can also vary with the age of the donor.

Neural stem cells or neural precursor cells can also be isolated from post-natal and adult tissues. Neural stem cells derived from post-natal and adult tissues are quantitatively equivalent with respect to their capacity to differentiate into neurons and glia, as well as in their growth and differentiation characteristics. However, the efficiency of in vitro isolation of neural stem cells from various post-natal and adult CNSs can be much lower than isolation of neural stem cells from fetal tissues, which harbor a more abundant population of neural stem cells. Nevertheless, as with fetal-derived neural v cells, the disclosed methods enable at least about 30% of neural progenitor cells derived from neonatal and adult sources to differentiate into neurons in vitro. Thus, post-natal and adult tissues can be used as described above in the case of fetal-derived neural stem cells.

In an embodiment, human fetal spinal tissue is dissected under a microscope. A region of tissue corresponding to the lower cervical/upper thoracic segments is isolated. The neural progenitors are isolated and then expanded on poly-D-lysine coated culture vessels in a media containing fibronectin and basic fibroblast growth factor (bFGF; FGF-2). Cells are expanded and then concentrated to the desired target cell density of about 10,000 cells per microliter in a medium free of preservatives and antibiotics. Concentrated cells may be used fresh for implantation or frozen for later use.

In another embodiment, the neural stem cells or neural precursor cells are derived from embryonic stem cells or induced pluripotent stem cells. As used herein, the term “embryonic stem cell” refers to a stem cell isolated from the developing embryo that can give rise to all of the cells of the body (e.g., cells of the ecto-, meso-, and/or endo-dermal cell lineages). The term “induced pluripotent stem cell,” as used herein, refers to a stem cell derived from a somatic cell (e.g., a differentiated somatic cell) that has a higher potency than the somatic cell. Embryonic stem cells and induced pluripotent stem cells are capable of differentiation into more mature cells. Methods employed for growing and differentiating embryonic or induced pluripotent stem cells into neural stem cells (NSCs) in vitro can, for example, be such as those described in Daadi et al., PLoS One. 3(2):e1644 (2008).

The polynucleotide sequence coding for the missing or deficient protein such as an enzyme is transfected into a neural stem cell using any standard methods or techniques. The polynucleotide for cytosine deaminase protein can be derived from bacteria, yeast, or other organisms.

A neural stem cell or neural precursor cell is said to be “genetically altered/engineered”, “transfected,” or “genetically transformed” when a polynucleotide has been transferred into the cell by any suitable means of artificial manipulation, or where the cell is a progeny of the originally altered cell that has inherited the polynucleotide. The polynucleotide will often comprise a transcribable sequence encoding a protein of interest, which enables the cell to express the protein at an elevated level. The genetic alteration is said to be “inheritable” if progeny of the altered cell has the same alteration.

There are several standard molecular biology techniques that may be used to regulate expression of an exogenous polynucleotide in a neural stem cell as disclosed herein. For example, different promoters may be used to regulate the level of expression of the growth factor and/or regulate which progeny of the neural stem cell will express the factor. For example, a human Ubiquitin C (UbC), PGK, or CAG promoter confers distinct levels of expression of the growth factor in the differentiated neuronal and glial progeny of the human neural stem cells disclosed herein. Additionally or alternatively, expression may be driven and confined to certain progeny of the neural stem cells. For example, a human synapsin promoter may be used to direct expression of a growth factor to the neuronal progeny of the neural stem cells.

The culture methods can be optimized to achieve long-term, stable expansion of an individual cell line of neural stem cells from different areas and ages of CNS development while maintaining their distinct progenitor properties. In one embodiment, neural stem cells or neural precursor cells can be cultured according to the methods set forth in U.S. Pat. Nos. 8,460,651, 8,236,299, 7,691,629, 5,753,506, 6,040,180, or U.S. Pat. No. 7,544,511, the entireties of which are incorporated by reference herein.

In an embodiment, the neural stem cells or neural precursor cells are concentrated in a solution such as the clinically usable hibernation or freezing solutions described above. In an embodiment, the neural stem cells or neural precursor cells are concentrated to an appropriate cell density, which can be the same or different from the cell density for administration of the cells. In an embodiment, the cell density for administration can vary from about 1,000 cells per microliter to about 1,000,000 cells per microliter depending upon factors such as the site of the injection, the minimum dose necessary for a beneficial effect, and toxicity side-effect considerations.

The neural stem cells or neural precursor cells may be concentrated to a density of about 1,000 to about 1,000,000 cells per microliter. In one embodiment, the neural stem cells or neural precursor cells are concentrated to a density of about 2,000 to about 80,000 NSCs per microliter. In another embodiment, about 5,000 to about 50,000 neural stem cells or neural precursor cells per microliter are used for effective treatment. In another embodiment, about 10,000 to 30,000 neural stem cells or neural precursor cells per microliter are used. In a preferred embodiment, the neural stem cells or neural precursor cells are concentrated to a density of about 70,000 neural stem cells per microliter.

In another embodiment, the neural stem cells or neural precursor cells are concentrated to a density of about 1,000 to about 10,000 cells per microliter, about 10,000 to about 20,000 cells per microliter, about 20,000 to about 30,000 cells per microliter, about 30,000 to about 40,000 cells per microliter, about 40,000 to about 50,000 cells per microliter, about 50,000 to about 60,000 cells per microliter, about 60,000 to about 70,000 cells per microliter, about 70,000 to about 80,000 cells per microliter, about 80,000 to about 90,000 cells per microliter, or about 90,000 to about 100,000 cells per microliter.

In another embodiment, the neural cells are concentrated to a density of about 100,000 to about 200,000 cells per microliter, about 200,000 to about 300,000 cells per microliter, about 300,000 to about 400,000 cells per microliter, about 400,000 to about 500,000 cells per microliter, about 500,000 to about 600,000 cells per microliter, about 600,000 to about 700,000 cells per microliter, about 700,000 to about 800,000 cells per microliter, about 800,000 to about 900,000 cells per microliter, or about 900,000 to about 1,000,000 cells per microliter.

The disclosure can be practiced using neural cells of any vertebrate species. Included are neural cells from humans as well as non-human primates, domestic animals, livestock, and other non-human mammals.

Certain neural precursor cells of this disclosure are obtained by culturing, differentiating, or reprogramming neural cells in a special growth environment that enriches cells with the desired phenotype (either by outgrowth of the desired cells, or by inhibition or killing of other cell types). These methods are WO 01/88104 PCT/US01/15861, applicable to many types of neural cells.

Typically, the differentiation takes place in a culture environment comprising a suitable substrate, and a nutrient medium to which the differentiation agents are added. Suitable substrates include solid surfaces coated with a positive charge, such as a basic amino acid, exemplified by poly-L-lysine, poly-D-lysine, and polyornithine.

Substrates can be coated with extracellular matrix components, exemplified by fibronectin. Other permissive extracellular matrixes include Matrigel® (extracellular matrix from Engelbreth-Holm-Swarm tumor cells) and laminin. Also suitable are combination substrates, such as poly-D-lysine combined with fibronectin, laminin, or both.

Optionally, the differentiated cells can be sorted based on phenotypic features to enrich for certain populations. Typically, this will involve contacting each cell with an antibody or ligand that binds to a marker characteristic of neural cells, followed by separation of the specifically recognized cells from other cells in the population. One method is immunopanning, in which a specific antibody is coupled to a solid surface. The cells are contacted with the surface, and cells not expressing the marker are washed away. The bound cells are then recovered by more vigorous elution. Variations of this are affinity chromatography and antibody-mediated magnetic cell sorting. In a typical sorting procedure, the cells are contacted with a specific primary antibody, and then captured with a secondary anti-immunoglobulin reagent bound to a magnetic bead. The adherent cells are then recovered by collecting the beads in a magnetic field.

The expression of tissue-specific gene products can also be detected at the mRNA level by Northern blot analysis, dot-blot hybridization analysis, or by reverse transcriptase initiated polymerase chain reaction (RT-PCR) using sequence-specific primers in standard amplification methods. See U.S. Pat. No. 5,843,780 for further details. Sequence data for the particular markers listed in this disclosure can be obtained from public databases such as GenBank (URL www.ncbi.nlm.nih.gov:80/entrez). Expression at the mRNA level is said to be “detectable” according to one of the assays described in this disclosure if the performance of the assay on cell samples according to standard procedures in a typical controlled experiment results in clearly discernible hybridization or amplification product. Expression of tissue-specific markers as detected; at the protein or mRNA level is considered positive if the level is at least two-fold, and preferably more than 10- or above that of a control cell, such as an undifferentiated pPS cell, a fibroblast, or other unrelated cell type.

Other methods of immortalizing cells are also contemplated, such as transforming the cells with DNA encoding myc, the SV40 large T antigen, or MOT-2 (U.S. Pat. No. 5,869,243, International Patent Applications WO 97/32972 and WO 01/23555). Transfection with oncogenes or oncovirus products is less suitable when the cells are to be used for therapeutic purposes. Telomerized cells are of particular interest in applications of this disclosure where it is advantageous to have cells that can proliferate and maintain their karyotype; for example, in pharmaceutical screening, and in therapeutic protocols where differentiated cells are administered to an individual in order to augment CNS function.

The neural cells according to this disclosure can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by -18- WO 01/88104 PCT/US01/15861 G. Morstyn W. Sheridan eds., Cambridge University Press, 1996; and Hematopoietic Stem Cell Therapy, E. D. Ball, J. Lister P. Law, Churchill Livingstone, 2000.

Also provided are neural stem cell lines or neural precursor cell lines including stable cell lines (e.g., progeny cells remain substantially constant in their karyotype, growth and differentiation characteristics) that express a protein such as an enzyme. Certain stable cell lines may express the protein for at least five, at least six, at least seven, at least eight, at least nine, at least 10, at least 15, at least 20, at least 25, or at least 30 generations.

In an embodiment, the neural stem cell or neural precursor cell expresses a lysosomal enzyme. In a further embodiment, the lysosomal enzyme is selected from the group consisting of: α-L-iduronidase, iuronate-2-sulfatase, N-sulfoglucoasmine sulfohydrolase, α-N-acetylgluscoaminidase, β-D-glucuronidase, β-glucosidase, sphingomyelinase, galactocerebrosidase, arylsulfatase A, α-galactosidase, β-galactosidase, hexosaminidase A and/or B, a-fucosidase, sulfatases, acid ceramidase, α- or β-D-mannosidase, N-aspartyl-β-glucosaminidase, a-fucosidase, a-acetylgalactosaminidase, neuraminidase, aspartoacylase, and cathepsin A.

In another embodiment, the neural stem cell or neural precursor cell expresses an antibody or fragment thereof.

The generalized structure of antibodies or immunoglobulin is well known to those skilled in the art. These molecules are heterotetrameric glycoproteins, typically of about 150,000 daltons, composed of two identical light (L) chains and two identical heavy (H) chains and typically referred to as full-length antibodies. Each light chain is covalently linked to a heavy chain by one disulfide bond to form a heterodimer, and the heterotrameric molecule is formed through a covalent disulfide linkage between the two identical heavy chains of the heterodimers. Although the light and heavy chains are linked together by one disulfide bond, the number of disulfide linkages between the two heavy chains varies by immunoglobulin isotype. Each heavy and light chain also has regularly spaced intrachain disulfide bridges. Each heavy chain has at the amino-terminus a variable domain (VH), followed by three or four constant domains (CH1, CH2, CH3, and CH4), as well as a hinge region between CH1 and CH2. Each light chain has two domains, an amino-terminal variable domain (VL) and a carboxy-terminal constant domain (CL). The VL domain associates non-covalently with the VH domain, whereas the CL domain is commonly covalently linked to the CH1 domain via a disulfide bond. Particular amino acid residues form an interface between the light and heavy chain variable domains that defines the antibody's epitope specificity (Chothia et al., 1985, J. Mol. Biol. 186:651-663). Variable domains are also referred to herein as variable regions.

Certain domains within the variable domains differ extensive among different antibodies, i.e., are “hypervariable.” These hypervariable domains contain residues that are directly involved in the binding and specificity of each particular antibody for its specific antigenic determinant (epitope). Hypervariability, both in the light chain and the heavy chain variable domains, is concentrated in three segments known as complementarity determining regions (CDRs) or hypervariable loops (HVLs). CDRs are defined by sequence comparison in Kabat et al., 1991, In: Sequences of Proteins of Immunological Interest, 5th Ed. Public Health Service, National Institutes of Health, Bethesda, Md., whereas HVLs (also referred to herein as CDRs) are structurally defined according to the three-dimensional structure of the variable domain, as described by Chothia and Lesk, 1987, J. Mol. Biol. 196: 901-917. These two methods result in slightly different identifications of a CDR. As defined by Kabat, CDR-L1 is positioned at about residues 24-34, CDR-L2 at about residues 50-56, and CDR-L3 at about residues 89-97 in the light chain variable domain; CDR-H1 is positioned at about residues 31-35, CDR-H2 at about residues 50-65, and CDR-H3 at about residues 95-102 in the heavy chain variable domain. The exact residue numbers that encompass a particular CDR will vary depending on the sequence and size of the CDR. Those skilled in the art can routinely determine which residues comprise a particular CDR given the variable region amino acid sequence of the antibody. The CDR1, CDR2, and CDR3 of the heavy and light chains therefore define the unique and functional properties specific for a given antibody.

The three CDRs within each of the heavy and light chains are separated by framework regions (FR), which contain sequences that tend to be less variable. From the amino terminus to the carboxy terminus of the heavy and light chain variable domains, the FRs and CDRs are arranged in the order FR1, CDR1, FR2, CDR2, FR3, CDR3, and FR4. The largely β-sheet configuration of the FRs brings the CDRs within each of the chains into close proximity to each other as well as to the CDRs from the other chain. The resulting conformation contributes to the antigen binding site (see Kabat et al., 1991, NIH Publ, No. 91-3242, Vol. I, pp. 647-669), although not all CDR residues are necessarily directly involved in antigen binding.

FR residues and Ig constant domains are not directly involved in antigen binding, but contribute to antigen binding and/or mediate antibody effector function. Some FR residues are thought to have a significant effect on antigen binding in at least three ways: by non-covalently binding directly to an epitope, by interacting with one or more CDR residues, and by affecting the interface between the heavy and light chains. The constant domains are not directly involved in antigen binding but mediate various Ig effector functions, such as participation of the antibody in antibody dependent cellular cytotoxicity (ADCC), complement dependent cytotoxicity (CDC) and antibody dependent cellular phagocytosis (ADCP).

The light chains of vertebrate immunoglobulins are assigned to one of two clearly distinct classes, kappa (κ) and lambda (λ), based on the amino acid sequence of the constant domain. By comparison, the heavy chains of mammalian immunoglobulins are assigned to one of five major classes, according to the sequence of the constant domains: IgA, IgD, IgE, IgG, and IgM. IgG and IgA are further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively. The subunit structures and three-dimensional configurations of the classes of native immunoglobulins are well known.

Antibodies encompass monoclonal antibodies (including full-length monoclonal antibodies), multispecific antibodies (e.g., bispecific antibodies), and antibody fragments such as variable domains and other portions of antibodies that exhibit a desired biological activity, e.g., bind a tumor-specific antigen.

It should be understood that monoclonal antibodies can be made by any technique or methodology known in the art, including, e.g., the hybridoma method (Kohler et al., 1975, Nature 256:495), or recombinant DNA methods known in the art (see, e.g., U.S. Pat. No. 4,816,567), or methods of isolation of monoclonal recombinantly produced using phage antibody libraries, using techniques described in Clackson et al., 1991, Nature 352: 624-628, and Marks et al., 1991, J. Molecular. Biology. 222: 581-597.

Chimeric antibodies consist of the heavy and light chain variable regions of an antibody from one species (e.g., a non-human mammal such as a mouse) and the heavy and light chain constant regions of another species (e.g., human) antibody and can be obtained by linking the DNA sequences encoding the variable regions of the antibody from the first species (e.g., mouse) to the DNA sequences for the constant regions of the antibody from the second (e.g., human) species and transforming a host with an expression vector containing the linked sequences to allow it to produce a chimeric antibody. Alternatively, the chimeric antibody also could be one in which one or more regions or domains of the heavy and/or light chain are identical with, homologous to, or variants of the corresponding sequence in a monoclonal antibody from another immunoglobulin class or isotype, or from a consensus or germline sequence. Chimeric antibodies can include fragments of such antibodies, provided that the antibody fragment exhibits the desired biological activity of its parent antibody, for example, binding to the same epitope (see, e.g., U.S. Pat. No. 4,816,567; and Morrison et al., 1984, Proc. Natl. Acad. Sci. USA 81: 6851-6855).

The term, “antibody fragment” refers to a portion of a full-length antibody in which a variable region or a functional capability is retained, for example, binding to a tumor-specific antigen. Examples of antibody fragments include, but are not limited to, a Fab, Fab′, F(ab′)2, Fd, Fv, scFv and scFv-Fc fragment, a diabody, a linear antibody, a single-chain antibody, a minibody, a diabody formed from antibody fragments, and multispecific antibodies formed from antibody fragments.

Full-length antibodies can be treated with enzymes such as papain or pepsin to generate useful antibody fragments. Papain digestion is used to produce two identical antigen-binding antibody fragments called “Fab” fragments, each with a single antigen-binding site, and a residual “Fc” fragment. The Fab fragment also contains the constant domain of the light chain and the CH domain of the heavy chain. Pepsin treatment yields an F(ab′)2 fragment that has two antigen-binding sites and is still capable of cross-linking antigen.

Fab′ fragments differ from Fab fragments by the presence of additional residues including one or more cysteines from the antibody hinge region at the C-terminus of the CH domain. F(ab′)2 antibody fragments are pairs of Fab′ fragments linked by cysteine residues in the hinge region. Other chemical couplings of antibody fragments are also known.

An “Fv” fragment contains a complete antigen-recognition and binding site consisting of a dimer of one heavy and one light chain variable domain in tight, non-covalent association. In this configuration, the three CDRs of each variable domain interact to define an antigen-binding site on the surface of the VH-VL dimer. Collectively, the six CDRs confer antigen-binding specificity to the antibody.

A “single-chain Fv” or “scFv” antibody fragment is a single chain Fv variant comprising the VH and VL domains of an antibody where the domains are present in a single polypeptide chain. The single-chain Fv is capable of recognizing and binding an antigen. The scFv polypeptide may optionally also contain a polypeptide linker positioned between the VH and VL domains in order to facilitate formation of a desired three-dimensional structure for antigen binding by the scFv (see, e.g., Pluckthun, 1994, In The Pharmacology of Monoclonal Antibodies, Vol. 113, Rosenburg and Moore eds., Springer-Verlag, New York, pp. 269-315).

A “diabody” refers to small antibody fragments with two antigen-binding sites, which fragments comprise a heavy chain variable domain (V.sub.H) connected to a light chain variable domain (V.sub.L) in the same polypeptide chain (V.sub.H-V.sub.L or V.sub.L-V.sub.H). Diabodies are described more fully in, e.g., Holliger et al. (1993) Proceedings of the National. Academy of Sciences of the United States of America 90: 6444-6448.

Other recognized antibody fragments include those that comprise a pair of tandem Fd segments (VH-CH1-VH-CH1) to form a pair of antigen binding regions. These “linear antibodies” can be bispecific or monospecific as described in, for example, Zapata et al. 1995, Protein English. 8(10): 1057-1062.

A “humanized antibody” or a “humanized antibody fragment” is a specific type of chimeric antibody that includes an immunoglobulin amino acid sequence variant, or fragment thereof, which is capable of binding to a predetermined antigen and that, comprises one or more FRs having substantially the amino acid sequence of a human immunoglobulin and one or more CDRs having substantially the amino acid sequence of a non-human immunoglobulin. This non-human amino acid sequence often referred to as an “import” sequence is typically taken from an “import” antibody domain, particularly a variable domain. In general, a humanized antibody includes at least the CDRs or HVLs of a non-human antibody, inserted between the FRs of a human heavy or light chain variable domain.

In another aspect, an antibody may comprise substantially all of at least one, and typically two, variable domains (such as contained, for example, in Fab, Fab′, F(ab′)2, Fabc, and Fv fragments), in which all, or substantially all, of the CDRs correspond to those of a non-human immunoglobulin, and specifically herein, all of the CDRs are mouse or humanized sequences as detailed herein below and all, or substantially all, of the FRs are those of a human immunoglobulin consensus or germline sequence. In another aspect, an antibody also includes at least a portion of an immunoglobulin Fc region, typically that of a human immunoglobulin. Ordinarily, the antibody will contain both the light chain as well as at least the variable domain of a heavy chain. The antibody may also include one or more of the CH1, hinge, CH2, CH3, and/or CH4 regions of the heavy chain, as appropriate.

An antibody can be selected from any class of immunoglobulins, including IgM, IgG, IgD, IgA and IgE, and any isotype, including IgG1, IgG2, IgG3, IgG4, IgA1 and IgA2. For example, the constant domain can be a complement fixing constant domain where it is desired that the humanized antibody exhibit cytotoxic activity, and the isotype is typically IgG1. Where such cytotoxic activity is not desirable, the constant domain may be of another isotype, e.g., IgG2.

The antibodies also may be conjugated to prodrugs. A “prodrug” is a precursor or derivative form of a pharmaceutically active substance that is less cytotoxic to tumor cells compared to the parent drug and is capable of being enzymatically activated or converted into the more active form. See, e.g, Wilman, 1986, “Prodrugs in Cancer Chemotherapy,” In Biochemical Society Transactions, 14, pp. 375-382, 615th Meeting Belfast; and Stella et al., 1985, “Prodrugs: A Chemical Approach to Targeted Drug Delivery,” In “Directed Drug Delivery,” Borchardt et al. (ed.), pp. 247-267, Humana Press. Useful prodrugs include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs, and optionally substituted phenylacetamide-containing prodrugs, 5-fluorocytosine and other 5-fluorouridine prodrugs that can be converted into the more active cytotoxic-free drug.

Examples of cytotoxic drugs that can be derivatized into a prodrug form include, but are not limited to, alkylating agents such as thiotepa and cyclosphosphamide; alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethylenethiophosphoramide, and trimethylolomelamine; acetogenins (especially bullatacin and bullatacinone); camptothecin (including the synthetic analogue topotecan); bryostatin; callystatin; CC-1065 (including its adozelesin, carzelesin, and bizelesin synthetic analogues); cryptophycines (particularly cryptophycin 1 and cryptophycin 8); dolastatin; auristatins (including analogues monomethyl-auristatin E and monomethyl-auristatin F); duocarmycin (including the synthetic analogues, KW-2189 and CBI-TMI); eleutherobin; pancratistatin; sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, and prednimustine; trofosfamide; uracil mustard; nitrosureas such as carmustine, chlorozotocin, fotemustine, lomustine, nimustine, and ranimustine; antibiotics such as the enediyne antibiotics (e.g., calicheamicin, especially calichemicin gamma1l and calicheamicin phil1; see for example e.g., Agnew, Chem. Intl. Ed. Engl., 33: 183-186; dynemicin, including dynemicin A; bisphosphonates, such as clodronate; esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (Adriamycin™) (including morpholino-doxorubicin, cyanomorpholino-doxorubicin, 2-pyrrolino-doxorubicin, and deoxydoxorubicin), epirubucin, esorubicin, idarubicin, marcellomycin, mitomycins such as mitomycin C, mycophenolic acid, nogalamycin, olivomycins, peplomycin, potfiromycin, puromycine, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogues such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adranals such as aminoglutethimide, mitotane, trilostane; folic acid replenishers such as frolinic acid; aceglatone; aldophosphamide glycoside; aminolevulinic acid; eniluracil; amsacrine; bestrabucil; bisantrene; edatraxate; defofamine; democolcine; diaziquone; elfomithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK®; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitabronitol; mitolactol; pipobroman; gacytosine; arabinoside (Ara-C); cyclophosphamide; thiotepa; taxoids, e.g., paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine (Gemzar™); 6-thioguanine; mercaptopurine; methotrexate; platinum analogues such as cisplatin and carboplatin; vinblastine; platinum; etoposide (VP-16); ifosfamide; mitoxantrone; vincristine; vinorelbine Navelbine™; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids, or derivatives of any of the above. Also included in this definition are anti-hormonal agents that act to regulate or inhibit hormone action on tumors such as anti-estrogens and selective estrogen receptor modulators (SERMs), including, for example, tamoxifen (including Nolvadex™) raloxifene, droloxifene, 4-hydroxytamoxifen, trioxifene, keoxifene, LY117018, onapristone, and toremifene (Fareston™); aromatase inhibitors that inhibit the enzyme aromatase, which regulates estrogen production in the adrenal glands, such as, for example, 4(5)-imidazoles, aminoglutethimide, megestrol acetate (Megace™), exemestane, formestane, fadrozole, vorozole (Rivisor™), letrozole (Femara™), and anastrozole (Arimidex™); and anti-androgens such as flutamide, nilutamide, bicalutamide, leuprolide, and goserelin; and pharmaceutically acceptable salts, acids, or derivatives of any of the above.

An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is distinguished from the nucleic acid molecule as it exists in natural cells.

In various aspects of the present disclosure, expression of the antibody may employ one or more control sequences, i.e., polynucleotide sequences necessary for expression of an operably linked coding sequence in a particular host organism. The control sequences suitable for use in prokaryotic cells include, for example, promoter, operator, and ribosome binding site sequences. Eukaryotic control sequences include, but are not limited to, promoters, polyadenylation signals, and enhancers.

A nucleic acid sequence is “operably linked” when it is placed into a functional relationship with another nucleic acid sequence. For example, a nucleic acid presequence or secretory leader is operably linked to a nucleic acid encoding a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers are optionally contiguous. Linking can be accomplished by ligation at convenient restriction sites. If such sites do not exist, synthetic oligonucleotide adaptors or linkers can be used.

### Methods of Treating a Deficiency of a Protein in the Central Nervous System

The present disclosure provides methods for disseminating a protein such as an enzyme or antibody in a central nervous system tissue by depositing neural stem cells or neural precursor cells that comprise a nucleotide that encodes the enzyme in a white matter tract, wherein the neural stem cells or neural precursor cells secrete the protein. Such methods may be used to treat a disease or disorder associated by a missing or deficient protein such as an enzyme in the central nervous system.

In an embodiment, the neural stem cells or neural precursor cells are administered to the brain in an effective amount. The amount of cells administered to the brain is that same for patients 4 years of age and up.

In an embodiment, the neural stem cell or neural precursor cell expresses a lysosomal enzyme. In a further embodiment, the lysosomal enzyme is selected from the group consisting of: α-L-iduronidase, iuronate-2-sulfatase, N-sulfoglucoasmine sulfohydrolase, α-N-acetylgluscoaminidase, β-D-glucuronidase, β-glucosidase, sphingomyelinase, galactocerebrosidase, arylsulfatase A, α-galactosidase, β-galactosidase, hexosaminidase A and/or B, a-fucosidase, sulfatases, acid ceramidase, α- or β-D-mannosidase, N-aspartyl-β-glucosaminidase, a-fucosidase, a-acetylgalactosaminidase, neuraminidase, aspartoacylase, and cathepsin A.

The neural stem cells or neural precursor cells are first implanted into one or more white matter tracts in a subject's central nervous system including, brain and/or spinal cord by a surgical means. Once implanted, the neural stem cells or neural precursor cells and their subsequent (progeny) progenitors migrate along the white matter tract and spread widely and disseminate across the brain/spinal cord tissue. The cells may stably reside and seamlessly integrate with the CNS tissue and continuously secrete the missing or deficient protein.

The present disclosure also provides methods of treating a disease or disorder that is due to a lack or deficiency of a protein in the central nervous system in a subject in need thereof by administering neural stem cells or neural precursor cells that express an enzyme that is deficient in the central nervous system of the subject to one or more white matter tracts in the central nervous system (e.g. one or more white matter tracts near the site of protein deficiency).

Also provided are methods of treating a disease or disorder that is due to a lack of a lysosomal enzyme in a brain in a subject in need thereof (e.g., a subject having MPS IIIA (MPS3a)) by administering neural stem cells or neural precursor cells that express an enzyme that is deficient in the brain of the subject to one or more white matter tracts in the brain.

Such methods may include administering a therapeutically effective amount of neural stem cells or neural precursor cells disclosed herein to a subject including, for example, by injection. In an embodiment, a subject treated with the disclosed neural stem cells or neural precursor cells is immunosuppressed prior to, during, and/or after administration of the neural stem cells or neural precursor cells.

In some embodiments, “treating” or “treatment” of a disease, disorder, or condition includes at least partially: (1) preventing the disease, disorder, or condition, i.e. causing the clinical symptoms of the disease, disorder, or condition not to develop in a mammal that is exposed to or predisposed to the disease, disorder, or condition but does not yet experience or display symptoms of the disease, disorder, or condition; (2) inhibiting the disease, disorder, or condition, i.e., arresting or reducing the development of the disease, disorder, or condition or its clinical symptoms; or (3) relieving the disease, disorder, or condition, i.e., causing regression of the disease, disorder, or condition or its clinical symptoms.

The terms “prevention,” “prevent,” “preventing,” “suppression,” “suppress,” “suppressing,” “inhibit” and “inhibition” as used herein refer to a course of action (such as administering a neural progenitor cell as disclosed herein) initiated in a manner so as to prevent, suppress or reduce, either temporarily or permanently, the onset of a clinical manifestation of the disease state or condition. Such preventing, suppressing or reducing need not be absolute to be useful.

In some embodiments, “effective amount,” as used herein, refers to the amount of neural stem cells or precursor cells that is required to confer a therapeutic effect on the subject. A “therapeutically effective amount,” as used herein, refers to a sufficient amount of neural progenitor cells being administered which will relieve to some extent one or more of the symptoms of the disease, disorder, or condition being treated. In some embodiments, the result is a reduction and/or alleviation of the signs, symptoms, or causes of a disease, or any other desired alteration of a biological system. For example, in some embodiments, an “effective amount” for therapeutic uses is the amount of the neural progenitor cells required to provide a clinically significant decrease in disease symptoms without undue adverse side effects. In some embodiments, an appropriate “effective amount” in any individual case is determined using techniques such as a dose escalation study. The term “therapeutically effective amount” includes, for example, a prophylactically effective amount. In other embodiments, an “effective amount” of neural progenitor cells is an amount effective to achieve a desired pharmacologic effect or therapeutic improvement without undue adverse side effects. In other embodiments, it is understood that an effective amount” or a therapeutically effective amount” varies from subject to subject due to variation in metabolism, age, weight, general condition of the subject, the condition being treated, the severity of the condition being treated, and the judgment of the prescribing physician.

The levels of a protein in the central nervous system may be increased by 5%, 10%, 15%, 20%, 25%, 30%, 40%, 50%, 60%, 70%, 80%, 90%, 100%, 150%, 200% or greater, including as compared to a subject's brain that is not administered a therapeutically effective amount of one or more of the human neural progenitor cells disclosed herein.

In an embodiment, the NSCs can be diluted with an acceptable pharmaceutical carrier. The term “pharmaceutically acceptable carrier” as used herein refers to a diluent, adjuvant, excipient, or vehicle with which the cells of the disclosure are administered and which is approved by a regulatory agency of the federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the neural progenitor cells and pharmaceutically acceptable carriers can be sterile. Water is a useful carrier when the cells are administered intravenously. Saline solutions and aqueous dextrose and glycerol solutions can also be employed as liquid carriers, particularly for injectable solutions.

Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH-buffering agents. The present compositions advantageously may take the form of solutions, emulsions, sustained-release formulations, or any other form suitable for use. The selection of a suitable carrier is within the skill of the ordinary artisan.

The NSCs in the disclosed methods can be derived from one site and transplanted to another site within the same subject as an autograft. Furthermore, the NSCs in the disclosed methods can be derived from a genetically identical donor and transplanted as an isograft. Still further, the NSCs in the disclosed methods can be derived from a genetically non-identical member of the same species and transplanted as an allograft. Alternatively, NSCs can be derived from a non-human origin and transplanted as a xenograft. With the development of powerful immunosuppressants, allograft and xenograft of non-human neural precursors, such as neural precursors of porcine origin, can be grafted into human subjects.

A sample tissue can be dissociated by any standard method. In one embodiment, tissue is dissociated by gentle mechanical trituration using a pipette and a divalent cation-free buffer (e.g., saline) to form a suspension of dissociated cells. Sufficient dissociation to obtain largely single cells is desired to avoid excessive local cell density.

In another embodiment, the neural stem cells or neural precursor cells can be delivered to a treatment area suspended in an injection volume of less than about 100 microliters per injection site. For example, in the treatment of glioblastoma in a human subject where multiple injections may be made, an injection volume of 0.1 and about 100 microliters per injection site can be used. In preferred embodiments, the NSCs can be delivered to a treatment area suspended in an injection volume of about 1 microliter per injection site.

In an embodiment, the disclosed methods include injecting neural stem cells or neural precursor cells at a cell density of about 1,000 to about 10,000 cells per microliter, about 10,000 to about 20,000 cells per microliter, about 20,000 to about 30,000 cells per microliter, about 30,000 to about 40,000 cells per microliter, about 40,000 to about 50,000 cells per microliter, about 50,000 to about 60,000 cells per microliter, about 60,000 to about 70,000 cells per microliter, about 70,000 to about 80,000 cells per microliter, about 80,000 to about 90,000 cells per microliter, or about 90,000 to about 100,000 cells per microliter into one or more white matter tracts in a subject's central nervous system including, for example, the subject's brain.

In some embodiments, the disclosed methods include injecting neural stem cells or neural precursor cells at a cell density of about 100,000 to about 200,000 cells per microliter, about 200,000 to about 300,000 cells per microliter, about 300,000 to about 400,000 cells per microliter, about 400,000 to about 500,000 cells per microliter, about 500,000 to about 600,000 cells per microliter, about 600,000 to about 700,000 cells per microliter, about 700,000 to about 800,000 cells per microliter, about 800,000 to about 900,000 cells per microliter, or about 900,000 to about 1,000,000 cells per microliter into one or more white matter tracts in a subject's central nervous system including, for example, the subject's brain.

In an embodiment, the disclosed methods include injecting neural stem cells or neural precursor cells at a cell density of about 5,000 to about 50,000 cells per microliter. In preferred embodiments, the disclosed methods include injecting neural stem cells or neural precursor cells at a cell density of about 70,000 cells per microliter.

In an embodiment, the disclosed methods include multiple injections of neural stem cells or neural precursor cells at a cell number per injection of about 5,000 to about 50,000 cells, about 50,000 to about 100,000 cells, about 100,000 to about 150,000 cells, about 150,000 to about 200,000 cells, about 200,000 to about 250,000 cells, about 250,000 to about 300,000 cells, about 300,000 to about 350,000 cells, about 350,000 to about 400,000 cells, about 400,000 to about 450,000 cells, or about 450,000 to about 500,000 cells introduced into one or more white matter tracts in a subject's central nervous system including, for example, the subject's brain.

Alternatively, the disclosed methods include multiple injections of neural stem cells or neural precursor cells at a cell number per injection of about 500,000 to about 1,000,000 cells, about 1,500,000 cells to about 2,000,000 cells, about 2,000,000 cells to about 2,500,000 cells, about 2,500,000 cells to about 3,000,000 cells, about 3,000,000 cells to about 3,500,000 cells, about 3,500,000 cells to about 4,000,000 cells, about 4,000,000 cells to about 4,500,000 cells, about 4,500,000 cells to about 5,000,000 cells, about 5,000,000 cells to about 5,500,000 cells, about 5,500,000 cells to about 6,000,000 cells, about 6,000,000 cells to about 6,500,000 cells, about 6,500,000 cells to about 7,000,000 cells, about 7,000,000 cells to about 7,500,000 cells, about 7,500,000 cells to about 8,000,000 cells, about 8,000,000 cells to about 8,500,000 cells, about 8,500,000 cells to about 9,000,000 cells, about 9,000,000 cells to about 9,500,000 cells, about 9,500,000 cells to about 10,000,000 cells, about 10,000,000 cells to about 10,500,000 cells, about 10,500,000 cells to about 11,000,000 cells, about 11,000,000 cells to about 11,500,000 cells, about 11,500,000 cells to about 12,000,000 cells, about 12,000,000 cells to about 12,500,000 cells, about 12,500,000 cells to about 13,000,000 cells, about 13,000,000 cells to about 13,500,000 cells, about 13,500,000 cells to about 14,000,000 cells, about 14,000,000 cells to about 14,500,000 cells, about 14,500,000 cells to about 15,000,000 cells, about 15,000,000 cells to about 15,500,000 cells, or about 15,500,000 cells to about 16,000,000 cells.

In some embodiments, the disclosed methods include multiple injections of neural stem cells or neural precursor cells with a total cell number of about 400,000 to about 800,000 cells, about 800,000 to about 1,200,000 cells, about 1,200,000 to about 1,600,000 cells, about 1,600,000 to about 2,000,000 cells, about 2,000,000 to about 2,400,000 cells, about 2,400,000 to about 2,800,000 cells, about 2,800,000 to about 3,200,000 cells, about 3,200,000 to about 3,600,000 cells, about 3,600,000 to about 4,000,000 cells, about 4,000,000 to about 5,000,000, or about 5,000,000 to about 10,000,000 cells introduced into one or more white matter tracts in a subject's central nervous system including, for example, the subject's brain.

In other embodiments, the disclosed methods include multiple injections of neural stem cells or neural precursor cells with a total cell number of about 1 million, 2 million, 3 million, 4 million, 5 million, 6 million, 7 million, 8 million, 9 million, 10 million, 11 million, 12 million, 13 million, 14 million, 15 million, 16 million, 17 million, 18 million, 19 million, 20 million, 21 million, 22 million, 23 million, 24 million, 25 million, 26 million, 27 million, 28 million, 29 million, 30 million, 31 million, 32 million, 33 million, 34 million, 35 million, 36 million, 37 million, 38 million, 39 million, 40 million, 41 million, 42 million, 43 million, 44 million, 45 million, 46 million, 47 million, 48 million, 49 million, 50 million, 51 million, 52 million, 53 million, 54 million, 55 million, 56 million, 57 million, 58 million, 59 million, 60 million, 61 million, 62 million, 63 million, 64 million, 65 million, 66 million, 67 million, 68 million, 69 million, 70 million, 71 million, 72 million, 73 million, 74 million, 75 million, 76 million, 77 million, 78 million, 79 million, 80 million, 81 million, 82 million, 83 million, 84 million, 85 million, 86 million, 87 million, 88 million, 89 million, 90 million, 91 million, 92 million, 93 million, 94 million, 95 million, 96 million, 97 million, 98 million, 99 million, or 100 million cells introduced into one or more white matter tracts in a subject's central nervous system including, for example, the subject's brain.

In another embodiment, the disclosed methods include multiple injections of neural stem cells or neural precursor cells with a total cell number of about 100 million to about 200 million, about 200 million to about 300 million, about 300 million to about 400 million, about 400 million to about 500 million, about 500 million to about 600 million, about 600 million to about 700 million, about 700 million to about 800 million, about 800 million to about 900 million, about 900 million to about 1 billion, about 1 billion to about 1.1 billion, about 1.1 billion to about 1.2 billion, about 1.2 billion to about 1.3 billion, about 1.3 billion to about 1.4 billion, about 1.4 billion to about 1.5 billion, about 1.5 billion to about 1.6 billion, about 1.6 billion to about 1.7 billion, about 1.7 billion to about 1.8 billion, about 1.8 billion to about 1.9 billion, or about 1.9 billion to about 2.0 billion cells introduced into one or more white matter tracts in a subject's central nervous system including, for example, the subject's brain.

The volume of media in which the expanded neural stem cells or neural precursor cells are suspended for delivery to a treatment area can be referred to herein as the injection volume. The injection volume depends upon the injection site and the degenerative state of the tissue. More specifically, the lower limit of the injection volume can be determined by practical liquid handling of viscous suspensions of high cell density as well as the tendency of the cells to cluster. The upper limit of the injection volume can be determined by limits of compression force exerted by the injection volume that are necessary to avoid injuring the host tissue, as well as the practical surgery time.

Any suitable device for injecting the cells into a desired area can be employed in the disclosed methods. In an embodiment, a syringe capable of delivering sub-milliliter volumes over a time period at a substantially constant flow rate is used. The cells can be loaded into the device through a needle or flexible tubing or any other suitable transfer device.

In another embodiment, the cells are injected at between about 1 and about 100 sites in one or more white matter tracts in a subject's central nervous system including, for example, the subject's brain. In an embodiment, the cells are injected at between about 10 and about 20 sites in one or more white matter tracts in a subject's central nervous system including, for example, the subject's brain. In an embodiment, the cells are injected at between about 20 to about 30 sites in one or more white matter tracts in a subject's central nervous system including, for example, the subject's brain. In an embodiment, the cells are injected at between about 30 to about 50 sites in one or more white matter tracts in a subject's central nervous system including, for example, the subject's brain. In an embodiment, the cells are injected at between about 50 to about 100 sites in one or more white matter tracts in a subject's central nervous system including, for example, the subject's brain. In an embodiment, the cells are injected at between about 100 to about 150 sites in one or more white matter tracts in a subject's central nervous system including, for example, the subject's brain. In an embodiment, the cells are injected at between about 150 to about 200 sites in one or more white matter tracts in a subject's central nervous system including, for example, the subject's brain. In an embodiment, the cells are injected at between about 200 to about 250 sites in one or more white matter tracts in a subject's central nervous system including, for example, the subject's brain. In an embodiment, the cells are injected at between about 250 to about 300 sites in one or more white matter tracts in a subject's central nervous system including, for example, the subject's brain. In an embodiment, the cells are injected at between about 300 to about 350 sites in one or more white matter tracts in a subject's central nervous system including, for example, the subject's brain. In an embodiment, the cells are injected at between about 350 to about 400 sites in one or more white matter tracts in a subject's central nervous system including, for example, the subject's brain. In an embodiment, the cells are injected at between about 400 to about 450 sites in one or more white matter tracts in a subject's central nervous system including, for example, the subject's brain. In an embodiment, the cells are injected at between about 450 to about 500 sites in one or more white matter tracts in a subject's central nervous system including, for example, the subject's brain.

At least two of the sites can be separated by a distance of approximately 1 mm to about 50 mm including, for example in one needle track. In another embodiment, the distance between injections sites is about 1 mm, 2 mm, 3 mm, 4 mm, 5 mm, 6 mm, 7 mm, 8mm, 9 mm, 10 mm, 11 mm, 12 mm, 13 mm, 14 mm, 15 mm, 16 mm, 17 mm, 18 mm, 19 mm, or 20 mm. In an embodiment, the distance between injection sites is about 400 to about 600 microns. In an embodiment, the distance between injections sites is about 100 to about 200 microns, about 200 to about 300 microns, about 300 to about 400 microns, about 400 to about 500 microns, about 500 to about 600 microns, about 600 to about 700 microns, about 700 to about 800 microns, about 800 to about 900 microns, or about 900 to about 1,000 microns. In an embodiment, the distance between injection sites is about 1,000 to about 2,000 microns, about 2,000 to about 3,000 microns, about 3,000 to about 4,000 microns, or about 4,000 to about 5,000 microns.

The neural stem cells or neural precursor cells may be injected into a white matter track at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 sites. In certain embodiments, 2 or more (e.g., 3, 4, 5, 6, 7, 8, 9, 10, 11, 12, 13, 14, 15, 16, 17, 18, 19, 20, or more) sites may be injected.

In one embodiment, compositions of the neural stem cells or neural precursor cells of the disclosure are formulated as an injectable formulation and comprise, for example, an aqueous solution or suspension suitable for delivery to the brain. When preparing the composition for injection, particularly for intracerebral delivery, a continuous phase can be present that comprises an aqueous solution of tonicity modifiers, buffered to a pH 6-8. The tonicity modifiers can comprise, for example, sodium chloride, glucose, mannitol, sorbitol, trehalose, glycerol, or other pharmaceutical agents that render osmotic pressure of the formulation isotonic with blood. Alternatively, when a larger quantity of the tonicity modifier is used in the formulation, it can be diluted prior to injection with a pharmaceutically acceptable diluent to render the mixture isotonic with blood.

In some embodiments of any of the aforementioned methods, the composition comprising neural stem cells or neural precursor cells is administered once. In some embodiments of any of the aforementioned methods, administration of an initial dose of the composition comprising neural progenitor cells is followed by the administration of one or more subsequent doses. Examples of dosing regimens (e.g., an interval between the first dose and one or more subsequent doses) that can be used in the methods of the disclosure include an interval of about once every week to about once every 12 months, an interval of about once every two weeks to about once every six months, an interval of about once every month to about once every six months, an interval of about once every month to about once every three months, or an interval of about once every three months to about once every six months. In some embodiments, administration is monthly, every two months, every three months, every four months, every five months, every six months, or upon disease recurrence.

The methods of the present disclosure may include administration of one or more immunosuppressive drugs prior to, concurrent with, or after the injection of the neural progenitor cells. In some embodiments, the neural progenitor cells and immunosuppressive drug may be co-administered. The neural progenitor cells and immunosuppressive drug that make up the therapy may be a combined dosage form or in separate dosage forms intended for substantially simultaneous administration.

The neural stem cells or neural precursor cells and immunosuppressive drug, may also be administered sequentially, with either the neural progenitor cells or immunosuppressive drug being administered by a regimen calling for multiple-step administration. Thus, a regimen may call for sequential administration of the neural progenitor cells and immunosuppressive drug with spaced-apart administration of the separate, active agents. The period between the multiple administration steps may range from, for example, a few minutes to several hours to days, depending upon the properties of the neural progenitor cells and immunosuppressive drug such as potency, solubility, bioavailability, plasma half-life and kinetic profile of the therapeutic compound, as well as depending upon the effect of food ingestion and the age and condition of the subject.

The neural progenitor cells and immunosuppressive drug, whether administered simultaneously, substantially simultaneously, or sequentially, may involve a regimen calling for administration of the neural progenitor cells by an intravenous, intraarterial, intracerebral or intraventricular route and the immunosuppressive drug by an oral route, a percutaneous route, an intravenous route, or an intramuscular route, or by direct absorption through mucous membrane tissues, for example. Whether the immunosuppressive drug is administered orally, by inhalation spray, rectally, topically, buccally (for example, sublingual), or parenterally (for example, subcutaneous, intramuscular, intravenous and intradermal injections, or infusion techniques), separately or together, each such therapeutic compound will be contained in a suitable pharmaceutical formulation of pharmaceutically acceptable excipients, diluents or other formulation components.

The present disclosure is further illustrated by the following examples, which should not be construed as limiting in any way. The materials and methods as used in the following experimental examples are described below.

### EXAMPLES

### Example 1: Generation of a Neural Stem Cell that Expresses SGSH

A cell line, HK532.SGSH, of stable adherent hNSC (conditionally immortalized with cMyc-ER) overexpressing hSGSH under CAG promoter was created using a lentiviral vector. The parental hkNSC was chosen for its exceptional migratory property as well as its robust expansion capacity under tight specifications for future cGMP manufacturing and its physiological innate properties (e.g., predictable differentiation into 50% neurons and 50% glia in vitro and in vivo).

Briefly, a p-o-p SGSH-expressing lentiviral vector is constructed using standard techniques. Next, a culture from a cGMP MCB vial of hkNSC is transduced with the p-o-p-grade SGSH-lentivirus. Typically, the transduction efficiency is 70-90% from an overnight infection at MOI 10-100. There will be no selection step. The transduced cells are then expanded from for 9 serial passages under non-GM P to generate 3-tiered research-grade cell banks at passage 3 (p3), 6 (p6), and 9 (p9). The scale of each bank will be limited to 20 T175 flasks for p3 and p6 (approximately 4×10⁸ cells/bank) and 60 T175 flasks for p9 (approximately 2.4×10⁹ cells).

As shown, the recombinant SGSH protein under the CAG promoter has been demonstrated to be expressed (about 10-fold higher than the wildtype) and secreted out of the cells with the enzyme activity high enough to be therapeutic.

The p9 cells were also evaluated for key properties (karyology, enzyme level, growth rate and differentiation). Briefly, the In vitro properties of hkNSC.SGSH cells that were studied over 9 serial passages includes: growth rate, telomere length, the ratio of neural progenitors, neurons, astrocytes, and/or oligodendrocytes, and the extent of neuronal maturation. Further, the in vitro properties of hkNSC.SGSH cells that were studied included the amount and activity of SGSH enzyme in the conditioned media from p3, p6, and p9 cultures.

### Example 2: Optimization of SGSH Expression, Secretion, and Endocytosis

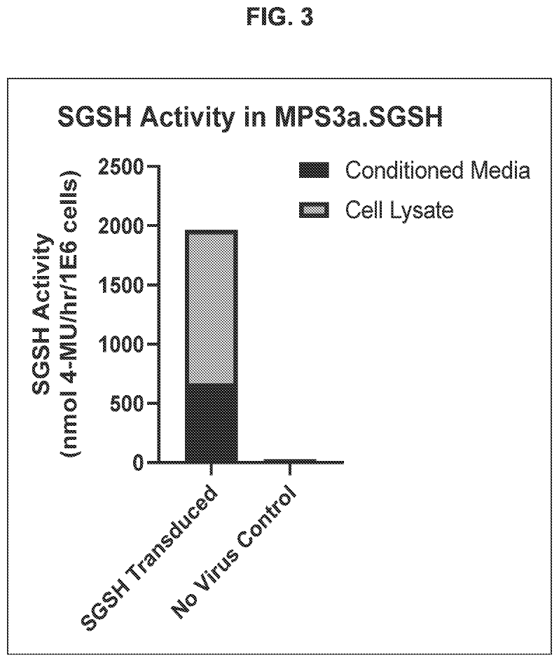

The entire CNS was normalized by delivering a sufficient amount of functionally active human N-sulfoglucosamine sulfohydrolase (hSGSH). A pilot scale lentivirus (LV) expressing hSGSH was generated to confirm that transduced hNSCs express and secrete functionally active hSGSH into the culture medium. The expression construct in LV was further optimized by (i) substituting the native secretory signal sequence with an artificial, more efficient signal sequence to increase SGSH secretion and (ii) reducing glycosylation sites by N264Q substitution to enhance endocytosis by target cells. A third generation LV was also utilized to increase its titer (VectorBuilder, Chicago). In order to evaluate the LV constructs, a surrogate hNSC line was established, wherein the line was derived from iPSC of a MPS IIIA patient (“MPS3a.hNSC”, GM27162, Corielle Institute), which shows undetectable background SGSH activity. Various LV transduction-enhancing polycations—polybrene, DEAE-dextran, Protamine S, and Retronectin—were tested with MPS3a.hNSC and the best GMP-compatible transduction conditions identified. Subsequently, MPS3a.hNSC was transduced with the optimized hSGSH LV at various multiplicity of infection. A seed bank of the optimized MPS3a.hNSC.SGSH line was cryopreserved, which was >90% viable upon thaw. This bank showed SGSH activity of >100-fold higher than physiological level, of which 34% was secreted into the conditioned media (CM) over 24 hours (FIG. 3). Incubation of non-transduced MPS3a.hNSC with the CM conferred 4.5% of the CM SGSH into the target cells over 24 hours.

### Example 3: Demonstration of Feasibility of Clinically Intended Cell Delivery Device and Procedure

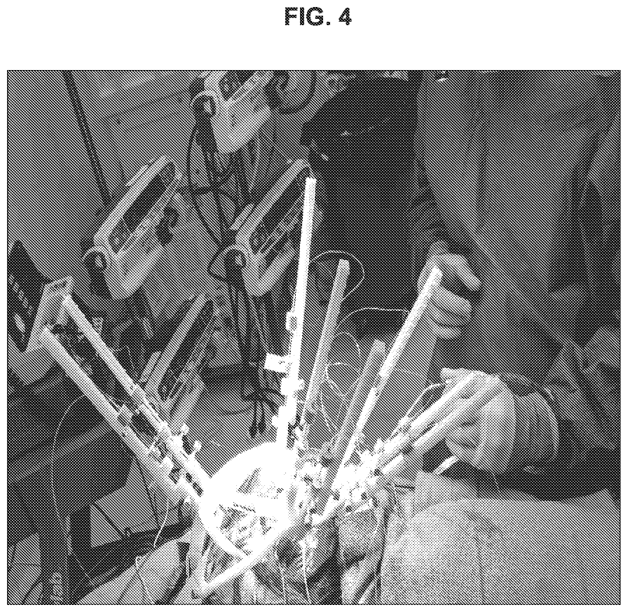

The delivery of sufficient SGSH-secreting cells into the CNS was tested, because such delivery is essential for strong therapeutic efficacy. Ideally, the cells should be placed at or near major white matter tracts so that they will travel on myelinated fibers, so that they disperse rapidly and widely across the brain and eventually down to the spinal cord. Hence, the surgical approach is to traverse the corona radiata, internal capsule, corpus callosum, and cerebellar tracts. Additionally, the clinically intended cell delivery components are: FDA-approved frameless stereotaxic platform (“StereoEEG”, FHC, Bowdoin, Me.), infusion cannula with detachable nylon tubing to hold the cell suspension (FHC), BD sterile disposable syringe (Becton Dickinson), and FDA-approved syringe pump (Alaris CC Guard Rail, Becton Dickinson). In a preliminary dog study with buffer only, it was demonstrated in healthy dogs (n=3) that the clinically intended device and cell infusion procedure are feasible and safe (FIG. 4). It was determined that the maximum tolerated infusion volume is approximately 10% of the brain volume.

### Example 4: In Vivo Assessment of hkNSC.SGSHr Cells

HkNSC.SGSHr obtained in Example 1 are tested for their ability to engraft in the brain and secrete SGSH using MPS3a.Rag2 genetically immunodeficient mouse.

Briefly, MPS3a.Rag2 (n=24) and their wildtype (normal) littermates (n=12) at postnatal day 1 (P1) are injected with 3 μL of vehicle or 0.24×10⁶ HkNSC.SGSHr cells in equal ratio into the striatum of each hemisphere (8) for 8-week survival. Next, the pups are genotyped at P0. Behavior performance is then evaluated at 8 weeks and compared between the groups. All of the normal (n=12) and half of the vehicle and cell cohorts of MPS3a.Rag2 are perfused transcardially with 4% paraformaldehyde (PFA)/PBS. The other half of MPS3a.Rag2 are dissected without perfusion for collection of snap-frozen fresh brains. Subsequently, brain homogenates from vehicle- (n=6) vs. cell-treated (n=6) MPS3a animals are compared for SGSH and HS levels and cell distribution and SGSH-expression domain in brain slices by IHC analyses. The vehicle- vs. cell-treated normal brains are then examined for tumorigenicity in hematoxylin eosin-stained brain slices.

### Example 5: Treatment of MPS3a Using a Neural Stem Cell that Expresses SGSH

HkNSC.SGSH cells are used to treat MPS3a by placing the cells into white matter tracts bilaterally across the corona radiata, internal capsule, and cerebellar tracts to achieve maximum cell spreading with minimum set of cannula penetrations. The cells continue to divide 2-3 cycles while migrating along the white matter tracts while the secreted SGSH enzyme diffuses through the interstitial fluid and is taken up by the neighboring host cells.

The maximum tolerated dose of administered cells is estimated to be approximately 1.6×10⁹ cells in 20 mL total at the cell concentration of 8×10⁷ cells/mL. This cell number is expected to expand to about 5.5-fold in 6 months in vivo to reach 8.8×10⁹ cells, which represents about 5% of the total human brain cells (estimated to be 1.7×10¹¹ cells with 1:1 composition of neurons:glia).

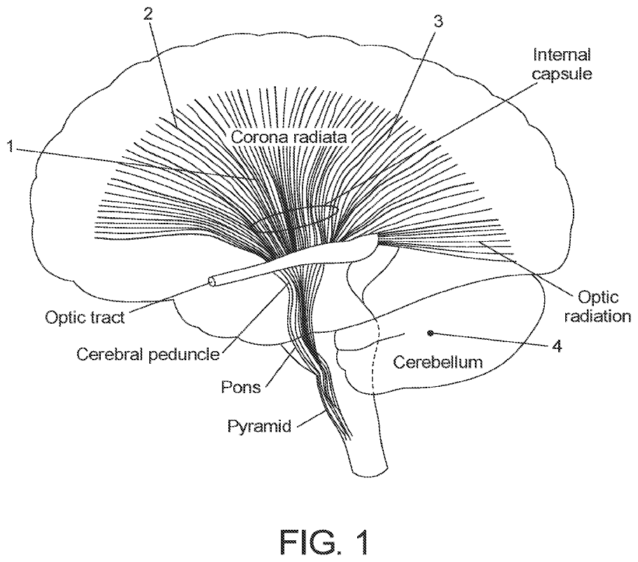

As illustrated in FIG. 1, eight injection tracks are used to deposit the cells into the white matter tracts of corona radiata, internal capsule, and cerebellum bilaterally. The surgery occurs in two stages: six tracks into the cerebrum in supine position, then two cerebellar tracks in prone position 2-8 weeks later. The stereotaxy for injection coordinates uses the standard frameless devices in current neurosurgical practice. The injection cannula for clinical studies is available commercially (FHC, Bowdain, Me.) and has been previously used with the planned cell density in stroke patients with no complications. Injection of the HkNSC.SGSH cells into the brain of a MPS3a patient reduces the amount of heparan sulfate (HS) accumulated in their brain.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 20. The other clauses can be presented in a similar manner.

Clause 1. A method for disseminating a protein in a central nervous system tissue, the method comprising depositing neural stem cells that comprise a nucleotide that encodes the protein in a white matter tract, wherein the neural stem cells secrete the enzyme.

Clause 2. The method of Clause 1, wherein the protein is a lysosomal enzyme.

Clause 3. The method of Clause 2, wherein the lysosomal enzyme is selected from the group consisting of: α-L-iduronidase, iuronate-2-sulfatase, N-sulfoglucoasmine sulfohydrolase, α-N-acetylgluscoaminidase, β-D-glucuronidase, β-glucosidase, sphingomyelinase, galactocerebrosidase, arylsulfatase A, α-galactosidase, β-galactosidase, hexosaminidase A and/or B, a-fucosidase, sulfatases, acid ceramidase, α- or β-D-mannosidase, N-aspartyl-β-glucosaminidase, a-fucosidase, a-acetylgalactosaminidase, neuraminidase, aspartoacylase, and cathepsin A.

Clause 4. The method of Clause 3, wherein the lysosomal enzyme is N-sulfoglucosamine sulfohydrolase (SGSH).

Clause 5. The method of Clause 1, wherein the neural stem cells are migratory neural stem cells.

Clause 6. The method of Clause 1, wherein the neural stem cells are capable of at least 60 cell doublings.

Clause 7. The method of Clause 1, wherein the neural stem cells are human neural stem cells.

Clause 8. The method of Clause 1, wherein the neural stem cells are adherent neural stem cells.

Clause 9. The method of Clause 1, wherein the central nervous system tissue is a brain.

Clause 10. The method of Clause 1, wherein the central nervous system tissue is a spinal cord.

Clause 11. The method of Clause 1, wherein the neural stem cells are derived from a fetal cortical tissue.

Clause 12. The method of Clause 1, wherein the neural stem cells are conditionally immortalized with cMyc-ER.

Clause 13. The method of Clause 1, wherein the neural stem cells are programmed to differentiate into neurons, oligodendrocytes, and astrocytes.

Clause 14. A method of treating a neurodegenerative disease that is due to a lack of a lysosomal enzyme in a brain in a subject in need thereof, the method comprising administering neural stem cells to one or more white matter tracts in the brain, wherein the neural stem cells express an enzyme that is deficient in the brain of the subject.

Clause 15. The method of Clause 14, wherein the neurodegenerative disease is MPS IIIA (MPS3a).

Clause 16. The method of Clause 14, wherein the enzyme is a lysosomal enzyme.

Clause 17. The method of Clause 16, wherein the lysosomal enzyme is α-L-iduronidase, iuronate-2-sulfatase, N-sulfoglucoasmine sulfohydrolase, α-N-acetylgluscoaminidase, β-D-glucuronidase, β-glucosidase, sphingomyelinase, galactocerebrosidase, arylsulfatase A, α-galactosidase, β-galactosidase, hexosaminidase A and/or B, a-fucosidase, sulfatases, acid ceramidase, α- or β-D-mannosidase, N-aspartyl-β-glucosaminidase, a-fucosidase, a-acetylgalactosaminidase, neuraminidase, aspartoacylase, and cathepsin A.

Clause 18. The method of Clause 17, wherein the lysosomal enzyme is N-sulfoglucosamine sulfohydrolase (SGSH).

Clause 19. The method of Clause 14, wherein the neural stem cells are deposited by intracerebral transplantation.

Clause 20. The method of Clause 14, wherein the central nervous system tissue is a brain.

Clause 21. The method of Clause 14, wherein the step of depositing the neural stem cells into the central nervous system tissue comprises injections of the cells into the white matter tracts of the corona radiata, internal capsule, and/or cerebellum bilaterally.

Clause 22. The method of Clause 14, wherein eight injection tracks are used to deposit the cells into the white matter tracts of corona radiata, internal capsule, and/or cerebellum bilaterally.

Clause 23. The method of Clause 22, wherein the injections are made in two stages.

Clause 24. The method of Clause 23, wherein the first stage comprises injections at six tracks into the cerebrum in supine position followed by injections at two cerebellar tracks about 2-8 weeks after the injections of the first stage.

Clause 25. The method of clause 15, wherein the neural stem cells comprise a vector comprising: (a) a human N-sulfoglucosamine sulfohydrolase (SGSH) coding sequence; and (b) an EF1A promoter, wherein the vector is a lentiviral vector, and wherein neural stem cells comprising the vector are characterized by activity of expressed SGSH protein that is between about 20- to about 300-fold higher than physiological activity of SGSH protein.

Clause 26. The method of clause 25, wherein the human SGSH coding sequence comprises an artificial secretory signal sequence, wherein the artificial secretory signal sequence increases SGSH secretion by between about 20% to about 200% as compared to the native secretory signal sequence of SGSH.

Clause 27. The method of clause 25, wherein the human SGSH coding sequence is a recombinant human SGSH coding sequence.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, the group for reasons of convenience and/or patentability. When any such inclusion or deletion occurs, the specification is deemed to contain the group as modified, thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

1. A method for disseminating a protein in a central nervous system tissue, the method comprising depositing neural stem cells that comprise a nucleotide that encodes the protein in a white matter tract, wherein the neural stem cells secrete the protein.

2. The method of claim 1, wherein the protein is a lysosomal enzyme.

3. The method of claim 2, wherein the lysosomal enzyme is selected from the group consisting of: α-L-iduronidase, iuronate-2-sulfatase, N-sulfoglucoasmine sulfohydrolase, α-N-acetylgluscoaminidase, β-D-glucuronidase, β-glucosidase, sphingomyelinase, galactocerebrosidase, arylsulfatase A, α-galactosidase, β-galactosidase, hexosaminidase A and/or B, a-fucosidase, sulfatases, acid ceramidase, α- or β-D-mannosidase, N-aspartyl-β-glucosaminidase, a-fucosidase, a-acetylgalactosaminidase, neuraminidase, aspartoacylase, and cathepsin A.

4. The method of claim 3, wherein the lysosomal enzyme is N-sulfoglucosamine sulfohydrolase (SGSH).

5. The method of claim 1, wherein the neural stem cells are migratory neural stem cells.

6. The method of claim 1, wherein the neural stem cells are capable of at least 60 cell doublings.

7. The method of claim 1, wherein the neural stem cells are human neural stem cells.

8. The method of claim 1, wherein the neural stem cells are adherent neural stem cells.

9. The method of claim 1, wherein the central nervous system tissue is a brain.

10. The method of claim 1, wherein the central nervous system tissue is a spinal cord.

11. The method of claim 1, wherein the neural stem cells are derived from a fetal cortical tissue.

12. The method of claim 1, wherein the neural stem cells are conditionally immortalized with cMyc-ER.

13. The method of claim 1, wherein the neural stem cells are programmed to differentiate into neurons, oligodendrocytes, and astrocytes.

14. A method of treating a neurodegenerative disease that is due to a lack of a lysosomal enzyme in a brain in a subject in need thereof, the method comprising administering neural stem cells to one or more white matter tracts in the brain, wherein the neural stem cells express an enzyme that is deficient in the brain of the subject.

15. The method of claim 14, wherein the neurodegenerative disease is MPS IIIA (MPS3a).

16. The method of claim 14, wherein the enzyme is a lysosomal enzyme.

17. The method of claim 16, wherein the lysosomal enzyme is α-L-iduronidase, iuronate-2-sulfatase, N-sulfoglucoasmine sulfohydrolase, α-N-acetylgluscoaminidase, β-D-glucuronidase, β-glucosidase, sphingomyelinase, galactocerebrosidase, arylsulfatase A, α-galactosidase, β-galactosidase, hexosaminidase A and/or B, a-fucosidase, sulfatases, acid ceramidase, α- or β-D-mannosidase, N-aspartyl-β-glucosaminidase, a-fucosidase, a-acetylgalactosaminidase, neuraminidase, aspartoacylase, and cathepsin A.

18. The method of claim 17, wherein the lysosomal enzyme is N-sulfoglucosamine sulfohydrolase (SGSH).

19. The method of claim 14, wherein the neural stem cells are deposited by intracerebral transplantation.

20. The method of claim 14, wherein the central nervous system tissue is a brain.

21. The method of claim 14, wherein the step of depositing the neural stem cells into the central nervous system tissue comprises injections of the cells into the white matter tracts of the corona radiata, internal capsule, and/or cerebellum bilaterally.

22. The method of claim 14, wherein eight injection tracks are used to deposit the cells into the white matter tracts of corona radiata, internal capsule, and/or cerebellum bilaterally.

23. The method of claim 22, wherein the injections are made in two stages.

24. The method of claim 23, wherein the first stage comprises injections at six tracks into the cerebrum in supine position followed by injections at two cerebellar tracks about 2-8 weeks after the injections of the first stage.

25. The method of claim 15, wherein the neural stem cells comprise a vector comprising:
(a) a human N-sulfoglucosamine sulfohydrolase (SGSH) coding sequence; and
(b) an EF1A promoter,
wherein the vector is a lentiviral vector, and
wherein neural stem cells comprising the vector are characterized by activity of expressed SGSH protein that is between about 20- to about 300-fold higher than physiological activity of SGSH protein.

26. The method of claim 25, wherein the human SGSH coding sequence comprises an artificial secretory signal sequence,
wherein the artificial secretory signal sequence increases SGSH secretion by between about 20% to about 200% as compared to the native secretory signal sequence of SGSH.

27. The method of claim 25, wherein the human SGSH coding sequence is a recombinant human SGSH coding sequence.
